(19) United States
(12) Patent Application Publication
(10) Pub. No.: US 2008/0004284 A1
(43) Pub. Date: Jan. 3, 2008

(54) METHOD FOR TREATING FIBROMYALGIA SYNDROME AND RELATED CONDITIONS

(76) Inventors: Ahmad H. RAHMAN, Port of Spain (TT); Gerald HART, Port of Spain (TT)

Correspondence Address: FROMMER LAWRENCE & HAUG, 745 FIFTH AVENUE- 10TH FL., NEW YORK, NY, 10151, US

(21) Appl. No.: 11/674,389

(22) Filed: Feb. 13, 2007

Related U.S. Application Data
(60) Provisional application No. 60/791,100, filed on Apr. 10, 2006.

(51) Int. Cl. A61K 31/495; A61P 25/00

(52) U.S. Cl. 514/252.12

(57) ABSTRACT

The present invention provides a method for treating certain rheumatic conditions such as fibromyalgia syndrome, chronic fatigue syndrome, myofascial pain syndrome, and Gulf War syndrome, among others, by administration of trimetazadine, along with related compositions and kits.

This application claims the benefit of U.S. Provisional Application No. 60/791,100, filed Apr. 10, 2006.

### FIELD OF THE INVENTION

The present invention relates generally to compositions and methods for the treatment of certain rheumatic conditions such as fibromyalgia syndrome (FMS), chronic fatigue syndrome (CFS), myofascial pain syndrome (MPS), and Gulf War Syndrome (GWS), among others.

### BACKGROUND OF THE INVENTION

Fibromyalgia is a syndrome characterized by chronic and intense generalized pain over portions of the body. The pain is not limited to muscle tissue and may also be experienced in the skin. FMS is estimated to affect 2-5% of the population, and associated symptoms often include fatigue, malaise, depression, anxiety, muscle tightness in the morning, muscle stiffness, and sleep disorders. FMS is characterized by a generalized heightened perception of sensory stimuli. Other symptoms may include headaches, facial pain, cognitive impairment, gastrointestinal complaints, frequent urination, diarrhea, constipation and dysmenorrhea.

Various theories exist as to possible causes of FMS, although no agreement exists within the medical community, and at present, the causes and mechanisms triggering the onset of FMS are not known. Fibromyalgia has been recognized only since the 1980s as a medical disorder, and its proper diagnosis is complicated by its diverse symptoms and the lack of definitive diagnostic tests, e.g., immunological, virological, pathological, and the like, for identifying the condition. In general, FMS is one of a number of poorly understood illnesses, including chronic fatigue syndrome (CFS), myofascial pain syndrome (MPS), and others. A number of medications have been shown to have some degree of effectiveness in randomized clinical trials of patients with fibromyalgia, including antidepressants such as amitripyline, duloxetine, fluoxetine, and paroxetine; muscle relaxants such as cyclobenzaprine; and certain apalgesics such as tramadol. Currently, no medication has been approved by the FDA for treating fibromyalgia, and FMS patients often suffer for years from widespread pain.

CFS is a disorder characterized by fatigue of an incapacitating nature lasting at least six months. CFS can affect virtually every major system in the body, including neurological, immunological, hormonal, gastrointestinal, and musculoskeletal (Friedberg, F., et al., “Understanding Chronic Fatigue Syndrome: An Empirical Guide to Assessment and Treatment”, Washington D.C.: Americal Psychological Association; 1998; Fukuda, K., et al., Ann Intern Med 1994; 121:953-9). In addition to experiencing severe chronic fatigue, patients often exhibit several of the following symptoms: substantial impairment in short term memory, sore throat, tender lymph nodes, muscle pain, multi-joint pain without swelling or redness, headaches, unrefreshing sleep, and post-exertional malaise. Similar to FMS, definitive diagnostic markers or laboratory tests are unavailable to distinguish CFS. Thus, clinical diagnosis is largely based on self-reported symptoms and behavioral criteria. Relatively few patients with CFS are reportedly cured; a review by Joyce et al. reports that fewer than 10% of patients return to pre-illness levels of functioning following treatment (Joyce, J.; et al., Q J Med 1997; 90: 223-33).

Many of the symptoms associated with CFS are also characteristic of other similar conditions, such as FMS and MPS. Several studies have suggested that CFS and FMS have many similarities (Buchwald D., Rheum Dis Clin North Am, 1996: 22:219-43; Goldenberg D L, J. Rheumatol 1988: 15:992-6). In fact, it has been estimated that 20% to 70% of patients with FMS meet the criteria for CFS and that about 35% to 75% of patients with CFS also have FMS (Buchwald, D., et al., Arch Intern Med 1994; 154:2049-53; Hudson, J I, et al., Am J Med 1992; 92:363-7). Typical treatment approaches for CFS include the administering of low doses of drugs directed to treatment of the symptoms experienced by the individual patient. Such drugs include tricyclic agents such as doxepin, amitriptyline, and nortriptyline, for improving sleep and relieving mild generalized pain; antidepressants such as fluoxetine, sertraline, and paroxetine, among others; anxiolytic agents to treat panic disorder such as alprazolam, clonazepam, and lorazepam; and nonsteroidal anti-inflammatory drugs (NSAIDs) for relieving pain and fever such as naproxen, ibuprofen, and piroxicam. Additionally, antihypotensive agents such as fludrocortisone and beta blockers such as atenolol have been prescribed. Again, although many different approaches have been used to treat CFS, no single therapeutic agent or combination of therapeutic agents has been found to be significantly effective in the treatment of CFS.

Chronic myofascial pain (CMP), also referred to as myofascial pain syndrome (MPS), is a condition involving localized myofascial pain caused by trigger points. Myofascial patients commonly radiate pain from trigger points which are considered to be active. A myofascial trigger point is a localized area that is starving for oxygen. This results in release of neuroreactive biochemicals which sensitize nearby nerves. The sensitized nerves then initiate the motor, sensory, and autonomic effects of myofascial trigger points by acting on the central nervous system. Myofascial trigger points can be identified and documented electrophysiologically. They may also be identified histologically by contraction knots. Trigger points in MPS patients may be latent (nonsymptomatic) or active (producing pain, at rest or with motion or loading to the muscle). Latent trigger points are typically activated by intense heat or cold, changing or damp weather, repetitive injury, and weekend athletic syndrome. Additional factors leading to vulnerability towards MPS include short leg syndrome, small hemipelvis, poor posture, prolonged immobility, vitamin and mineral deficiencies, endocrine dysfunctions, intense emotional stress, and poor work habits (Travell, J. F. and Simmons, D. G.: Myofascial Pain & Dysfunction: The Trigger Point Manual, VI &2, Baltimore, Williams & Wilkins). Myofascial pain syndrome is often misdiagnosed as fibromyalgia. Current treatment approaches include physical therapy, trigger point injection with a local anesthetic, and the administration of drugs such as NSAIDs (e.g., ibuprofen), tricyclic antidepressants (e.g., amitriptyline), muscle relaxants (e.g., cyclobenzaprine), non-narcotic analgesics (e.g., tramadol), and anticonvulsants (e.g., gabapentin). Similar to the above conditions, while certain treatments can provide a degree of relief for certain patients, there currently exists no widely-accepted therapeutic approach for the effective treatment of MPS.

Often, many of the above conditions are misdiagnosed, or even undiagnosed. Currently existing therapies for the treatment of FMS, CFS, and MPS, have been of a limited, if any, well-accepted degree of success. Patients suffering from one or more of the foregoing conditions typically endure significant disabilities in terms of physical, occupational, and social functioning. Based upon the on-going research focused on these conditions, it can be seen that there is a need for an effective therapy for the treatment of FMS, CFS, MPS, and similar disorders. In particular, there is a need for compositions and treatments effective to significantly ameliorate or ideally, eliminate, one or more symptoms associated with one or more of the above conditions. It is believed that the present invention meets this need.

### SUMMARY OF THE INVENTION

Accordingly, in one aspect, the invention provides a method for the treatment of conditions such as fibromyalgia syndrome, chronic fatigue syndrome, and myofascial pain syndrome, among others.

The inventors arrived at the present discovery (forming the basis of the invention) in a completely unexpected fashion. While treating a subject suffering from a cardiac condition with an anti-anginal agent, the inventors discovered that, within a short time after commencement of treatment, that same subject, also suffering from fibromyalgia syndrome, experienced a surprising and remarkable improvement in all symptoms associated with his fibromyalgia. This effect was later further confirmed in additional in vivo studies of fibromyalgia patients for whom currently existing treatments for fibromyalgia had previously been either minimally or completely ineffective.

Thus, in one aspect, the invention is directed to a method of treating a rheumatic condition selected from the group consisting of fibromyalgia syndrome (FMS), chronic fatigue syndrome (CFS), myofascial pain syndrome (MPS), and Gulf War syndrome (GWS). The method comprises administering to a mammalian subject suffering from such condition a therapeutically effective amount of trimetazidine over a duration of time effective to result in a diminution of one or more major symptoms associated with the condition being treated.

In one or more embodiments of the method, the administering is over a duration of time effective to result in at least a 50% improvement in one or more major symptoms associated with the particular rheumatic condition.

In yet one or more additional embodiments, the administering is over a duration of time effective to result in at least a 90% improvement in one or more symptoms associated with the particular rheutmatic condition.

In yet another aspect, provided is a method of treating FMS comprising administering to a mammalian subject suffering from FMS a therapeutically effective amount of trimetazidine over a duration of time effective to result in a diminution of one or more major symptoms associated with said fibromyalgia, wherein one of said symptoms is widespread pain of at least three anatomical sites of the subject's body.

In one or more preferred embodiments, the administering is over a duration of time effective to result in at least a 50% reduction in the widespread pain experienced by the subject, preferably over a duration of time effective to result in at least a 90% reduction in the widespread pain.

In one or more embodiments, a therapeutically effective amount of trimetazidine ranges from approximately 5 milligrams to 210 milligrams daily, preferably from approximately 20 milligrams to 100 milligrams daily, and even more preferably from approximately 40 milligrams to 80 milligrams daily.

In yet one or more further embodiments, trimetazadine is administered orally or parenterally.

In yet another embodiment, administering trimetazidine over a duration of 72 hours is effective to result in at least a 50% reduction in widespread pain experienced by a subject suffering from FMS.

In yet a further embodiment, administering trimetazadine is effective to result in a diminution of one of more symptoms selected from the group consisting of: trigger point pain and/or tenderness, depression, dizziness, impaired concentration, irritable bowel syndrome, headache, fatigue, and sleep disturbance in a subject suffering from FMS.

Preferably, such diminution of one or more symptoms is an improvement of at least 50% of one or more symptoms selected from the group consisting of: trigger point pain and tenderness, depression, dizziness, impaired concentration, irritable bowel syndrome, headache, fatigue, and sleep disturbance.

In yet another aspect, provided is a kit comprising trimetazidine in packaged form, and instructions for administering trimetazidine for treatment of a condition selected from the group consisting of fibromyalgia syndrome, chronic fatigue syndrome, myofascial syndrome, and Gulf War syndrome.

In one or more embodiments of the invention;, trimetazidine is in a form selected from the group consisting of a tablet, syrup, suspension, and capsule.

The invention also provides, in yet another aspect, the use of trimetazidine for treatment of a condition selected from fibromyalgia syndrome, chronic fatigue syndrome, myofascial pain syndrome, and Gulf War syndrome.

Further, the invention is directed to the use of trimetazidine for the manufacture of a medicament for treating a condition selected from fibromyalgia syndrome, chronic fatigue syndrome, myofascial syndrome, and Gulf War syndrome.

Each of the herein-described features of the invention is meant to apply equally to each and every embodiment as described herein, unless otherwise indicated.

Additional objects, advantages and novel features of the invention will be set forth in the description that follows, and in part, will become apparent to those skilled in the art upon reading the following, or may be learned by practice of the invention.

### DETAILED DESCRIPTION OF THE INVENTION

### Definitions

Before describing the present invention in detail, it is to be understood that this invention is not limited to particular formulations of trimetazidine, administration modes, and the like, as such may vary, as will be apparent from the accompanying description.

It must be noted that, as used in this specification and the intended claims, the singular forms “a,” “an,” and “the” include plural referents unless the context clearly dictates otherwise. Thus, for example, reference to “a drug” includes a single drug as well as two or more of the same or different drugs, reference to “an optional excipient” refers to a single optional excipient as well as two or more of the same or different optional excipients, and the like.

In describing and claiming the present invention, the following terminology will be used in accordance with the definitions described below.

“Pharmaceutically acceptable excipient or carrier” refers to an excipient that may optionally be included in the compositions of the invention and that causes no significant adverse toxicological effects to the patient.

“Pharmacologically effective amount,” “physiologically effective amount,” and “therapeutically effective amount” are used interchangeably herein to mean the amount of a drug or drug-combination that is needed to provide a desired level of drug in the bloodstream or in the target tissue. The precise amount will depend upon numerous factors, e.g., the particular drug or drugs employed, the components and physical characteristics of the therapeutic composition, intended patient population, individual patient considerations, and the like, and can readily be determined by one skilled in the art, based upon the information provided herein.

“Pharmaceutically acceptable salt” includes, but is not limited to, amino acid salts, salts prepared with inorganic acids, such as chloride, sulfate, phosphate, diphosphate, hydrobromide, hydrochloride, and nitrate salts, or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium). In a preferred embodiment, trimetazidine is provided as a hydrochloride or dihydrochloride salt.

“Active molecule” or “active agent” as described herein includes any agent, drug, compound, composition of matter or mixture which provides some pharmacologic, often beneficial, effect that can be demonstrated in-vivo or in vitro. This includes foods, food supplements, microbiologicals, nutrients, nutriceuticals, drugs, vaccines, antibodies, vitamins, and other beneficial agents. As used herein, the terms further include any physiologically or pharmacologically active substance that produces a localized or systemic effect in a patient.

“Substantially” or “essentially” means nearly totally or completely, for instance, 95% or greater of some given quantity. For example, a substantial elimination of one or more symptoms or clinical indicators, e.g., of fibromyalgia syndrome or chronic fatigue syndrome, etc., means a reduction in severity of 95% or more of a symptom such as widespread pain, fatigue, irritable bowel syndrome, insomnia, cognitive and memory impairment, morning stiffness, dizziness, irritability, depression, as assessed by any clinically acceptable method, or an improvement of at least 95% of a given clinical indicator.

A “diminution” of one or more symptoms or clinical indicators, e.g., a major symptom associated with fibromyalgia syndrome, chronic fatigue syndrome, myofascial pain syndrome, or any other condition treatable in accordance with the present invention, means a measurable reduction in the severity of such one or more symptoms, as assessed by any clinically acceptable method, or a measurable improvement of a given clinical indicator, as assessed by a skilled clinician.

The term “patient,” refers to a living mammalian organism suffering from or prone to a condition that can be prevented or treated by administration of a drug or combination of drugs of the invention, and includes both humans and animals.

“Optional” or “optionally” means that the subsequently described circumstance may or may not occur, so that the description includes instances where the circumstance occurs and instances where it does not.

The term “treating” includes preventing, essentially eradicating, or ameliorating one or more major symptoms associated with the condition being treated, e.g., FMS, CFS, MFS, and the like. Thus, treatment may be accomplished, for example, when the patient reports decreased severity, duration, or recurrence of pain, a reduction in the number of anatomical sites affected by pain, a reduction in the number of tender points or trigger points, etc.

Method of Treatment—Overview

As described previously, the inventors have discovered trimetazidine to be uniquely effective in treating the symptoms of FMS, and in treating the widespread spectrum of FMS-associated symptomatology (e.g., CFS, MPS, MCS (multiple chemical sensitivities), and the like).

In each of the instances in which the applicants have administered trimetazidine to a patient suffering from FMS, and experiencing one or more of the following symptoms: widespread pain, tender points, fatigue, irritable bowel syndrome, insomnia, cognitive and memory impairment, morning stiffness, dizziness, depression, acid reflux, following a course of treatment of at least four weeks, the patient has reported at least a remarkable and measurable reduction in widespread pain, and in some instances, has described substantially complete alleviation of widespread pain, if not of all of the symptoms. See, for example, Examples 1-4 herein, which demonstrate a significant FMS symptom-modifying response upon administration of trimetazidine.

Briefly, in studies of eleven human subjects, each reported a remarkable improvement in widespread pain experienced by the subjects prior to treatment. In sum, within only 72 hours of commencement of treatment with trimetazidine, each of the subjects reported a marked reduction in widespread pain, and where applicable, tender point pain. More specifically, each of the subjects treated in accordance with the invention reported, on average, a 90% reduction in widespread pain within 72 hours of commencing treatment. Further, all patients reported significant improvement of minor symptoms within the same period. Follow-up examinations at either four months or four weeks of treatment revealed, for all patients, remarkably sustained relief or even improved relief of widespread pain, e.g., 90-95% improvement. Thus, it can be seen that the method of the present invention is remarkably effective in the treatment of rheumatic conditions such as FMS and the like.

Method of Diagnosis

Fibromyalgia. Fibromyalgia, also referred to as fibromyalgia syndrome, is part of a spectrum of chronic widespread pain of unknown origin (Bennett, R., Curr Opin Rheumatol., 1998, March 10(2): 95-103). According to Bennett (ibid), the prevalence of chronic widespread pain is several times higher than fibromyalgia as defined by the 1990 American College of Rheumatology guidelines. Thus, the method of the present invention is directed to treating not only fibromyalgia, but also to treating the spectrum of chronic widespread pain having fibromyalgia-associated symptomatology, e.g., CFS, MPS, MCS, and the like. Although pain and tenderness are its defining features, fatigue, sleep disturbance, depression, and poor concentration are also common.

Fibromyalgia can be diagnosed in a clinical setting by counting the number of tender points a patient has. (Wolfe, F., Ann Rheum Dis 1997; 56:268-272 (April)). In 1990, the American College of Rheumatology established criteria for diagnosing fibromyalgia, which includes the presence of 11 or more tender points and widespread pain of at least three months' duration (Wolfe, F., et. al., The American College of Rheumatology 1990 Criteria for the Classification of Fibromyalgia: Report of the Multicenter Criteria Committee. Arthritis Rheum 1990; 33:160-172 (Medline)). Such pain is typically present in all four quadrants of the body, i.e., on both the left and right side of the body and above and below the waist.

In assessing a subject for FMS, the patient is typically examined by undergoing a count of tender points using the 18 specified sites specified in the American College of Rheumatology 1990 Classification Criteria, ACRCC (ibid). Tender point data are reported as a count of positive test sites.

Pain threshold may be assessed using a dolorimetry examination (a pressure algometer). Such an examination is performed at the trapezii, knees, lateral epicondyle, and second rib. The examiner places the rubber tip of the dolorimeter on the examination site and gradually increases the pressure (e.g., at a rate of approximately 1 kg/cm² per second). The patient is asked to report the moment when the sensation at the site changes from that of pressure to that of pain. The force is then recorded at that point. The overall dolorimetry score is the mean of the sites examined. Among persons with FMS in a population survey, mean dolorimetry scores were approximately 2.7 kg/cm² (Wolfe, F., et al., Arthritis Rheum 1995; 38:19-28), while a median value in the normal population for women is 4.25 kg/cm² and 6.0 kg/cm² for men (Wolfe, F., et al, J. Rheumatol 1995; 22:151-156).

In evaluating a subject for FMS, a questionnaire is also typically used. An exemplary questionnaire is The Clinical Health Assessment Questionnaire (CLINHAQ) (Wolfe, F., Rheumatoid Arthritis: Pathogenesis, Assessment, Outcome, and Treatment. New York: Marcel Dekker, 1994; 463-514). This questionnaire contains self reports for the Health Assessment. Questionnaire (HAQ) disability index (Fries, J F, et al., Arthritis Rheum 1980; 23:137-145), arthritis impact measurement scales (AIMS) anxiety and depression index.(Hawley, D J, et al., J. Rheumatol 1993; 20:2025-203), visual analogue scale (VAS) pain, VAS global severity, VAS gastrointestinal symptoms, VAS sleep problems, VAS fatigue, satisfaction with health and patient estimate of health status. In 1996, the helplessness subscale of the rheumatology attitudes index (RAI) was added to the CLINHAQ (deVellis, R F, et al. J. Rheumatol 1993; 20:866-869). The variables contained in this questionnaire consider factors that are thought to be of major importance in fibromyalgia (Burckhardt, C S, et al., J. Rheumatol 1991; 18:728-733; Simms, R W, et al., J. Rheumatol 1991; 18:1558-1563).

Since there are no definite etiologic markers used for the diagnosis of FMS, approaches such as the above are typically used in diagnosing a patient with FMS, along with laboratory tests used to exclude the possibility of other disorders that could cause similar symptoms.

A study by Wolfe (Wolfe, F., Ann Rheum Dis 1997; 56:268-271), reports that FMS tenderness and symptoms are part of a continuum, and that, in a broad sense outside of a clinical setting, there is no discrete point where FMS does or does not exist, and that it is important to recognize the distress symptoms—whether or not the patient reaches the fibromyalgia diagnostic threshold as set forth in the ACRCC. For instance, alternative less stringent criteria for a positive diagnosis of FMS include, as part of the major criteria, generalized pain or stiffness of at least three anatomical sites for at least three months, and the existence of six or more tender points.

Thus, in accordance with the above, trimetazidine is useful in the treatment of FMS and of syndromes similar to fibromyalgia. That is to say, in one aspect, the invention encompasses the administering of trimetazidine for the treatment FMS, where the FMS patient is one that has experienced as major symptoms associated with FMS: (i) widespread pain associated with at least three anatomical sites of a duration of at least three months, and (ii) five or more tender points rather than the absolute 11 or more tender points defined by the ACRCC, even if such widespread pain does not fall within the clinical diagnosis of FMS. That is to say, treatment for FMS in accordance with the present invention includes treatment of subjects having a history of widespread pain of at least three months' duration and having fewer symptoms or tender points than 11, e.g., the patient may have 5 tender points, or 6 tender points, or 7 tender points, or 8 tender points, or 9 tender points, or even 10 points, or 11 tender points or more. Minor symptoms, which may also be treated in accordance with the invention, i.e., prevented, ameliorated, or eradicated, include fatigue, irritable bowel syndrome, sleep disorder, chronic headaches, jaw pain, cognitive or memory impairment, post-exertional malaise and muscle pain, morning stiffness, menstrual cramping, numbness and tingling sensations, dizziness, and skin and chemical sensitivities.

Treatment of any one or more of the major or minor symptoms described above, for this and related rheumatic conditions described herein, includes an identifiable diminution or amelioration of one or more symptoms or clinical indicators associated with fibromyalgia syndrome, chronic fatigue syndrome, myofascial pain syndrome, or any other condition treatable in accordance with the present invention, i.e., a measurable reduction in the severity of such one or more symptoms, as assessed by any clinically acceptable method, or a measurable improvement of a given clinical indicator, e.g., as assessed by skilled clinician. Treatment with trimetazidine is therefore effective to result in a measurable improvement in one or more symptoms associated with the subject rheumatic condition. Thus, treatment with trimetazidine may be effect to result in an improvement of at least about 20%, preferably at least about 30%, more preferably at least about 40%, even more preferably at least about 50%, and even more preferably at least about 60%, and even more preferably, at least about 70%, and even more preferably at least about 80%, even more preferably at least about 90%, and most preferably, at least about 95% or greater in a given major or minor symptom.

Chronic Fatigue Syndrome. Chronic fatigue syndrome is typically diagnosed using the Center for Disease Control and Prevention (CDC) 1994 guidelines published in the Annuals of Internal Medicine 121 (12):953-959: To meet these criteria, patients must have severe, unexplained fatigue that is not relieved by rest, which can cause disability and which has an identifiable onset. It should be persistent or relapsing fatigue that lasts for at least six or more consecutive months. In accordance with the present invention, persistent fatigue such as the above is considered a major symptom of CFS. According to the CDC guidelines, patients must also exhibit four or more of the following symptoms, considered herein as minor or secondary symptoms: impaired memory or concentration, tender cervical or axillary lymph nodes in the neck region, sore throat, muscle pain, multi-joint pain (but not arthritis), new onset headaches (of a new type, pattern or severity), unrefreshing sleep, and post-exertional malaise. As in the case of FMS, patients should be clinically evaluated to exclude other conditions that could be the cause of the above symptoms.

In many cases, FMS and CFS are co-existing conditions. It has been estimated that 20% to 70% of patients with FMS meet the criteria for CFS and that about 35% to 75% of patients with CFS also have FMS (Jason, L., et al., Psychosomatic Medicine 62:655-663 (2000)).

Illustrative measures for assessing CFS are as follows. The Fatigue Scale, as originally used in a hospital-based case study (Wessely, S., et al., J. Neurol Neurosurg Psychiatry 1989; 52: 940-8) and further refined by Chalder et al. (J. Psychosom Med 1993; 37:147-53), can be used to assess fatigue experienced by a subject. The fatigue scale is an eleven-item scale that has responses rated on a four-option continuum: the total score ranges from 0 to 33 (with a higher score indicating greater fatigue).

In combination with the above, one can optionally use a psychiatric interview such as the SCID (Structured Clinical Interview for DSM-IV Axis I Disorders, DSM-IV. Diagnostic and statistical manual of mental disorders. 4^{th} ed. Washington D.C.: American Psychiatric Association; 1994).

Additionally, to assess CFS, one can administer a medical questionnaire such as The Chronic Fatigue Questionnaire (Komaroff A L, et al., Am J Med 1996; 100:56-64) to assess symptoms related to CFS, as well as rule out exclusionary medical conditions.

Also, a detailed physical examination is typically conducted to rule out other possible medical conditions, including an 18-tender point examination as described above. Laboratory tests typically include one or more of the following: chemical screen (glucose, calcium, electrolytes, uric acid, liver function parameters, and renal function parameters), complete blood count with differential and platlet counts, T4 and thyroid-stimulating hormone, erythrocyte sedimentation rate, arthritic profile (e.g. rheumatoid factor and antinuclear antibody), hepatitis B surface antigen, creatine phosphokinase, an HIV screen and urinalysis.

Myofascial Pain Syndrome. Myofascial pain syndrome (also known as regional pain syndrome) has no uniformly accepted definition but is characterized as a regional muscle pain syndrome accompanied by trigger points (TPs) and their associated reflexes. A trigger point is a hyperirritable spot within a taut band of skeletal muscle or muscle fascia which is painful on compression and gives rise to characteristic referral pain patterns, tenderness and autonomic phenomena. A trigger point maybe active or latent. In accordance with the present invention, the existence of one or more trigger points is considered a major symptom of MPS.

Although there are no clear clinical criteria for diagnosing MPS, to diagnose an active myofascial trigger point, one looks for the following (Travell, J. F., Simons, D. G., Myofascial Pain and Dysfunction: The Trigger Point Manual. Baltimore; Williams & Wilkint, Volume 1, 1983: 18-19): (1) a history of sudden onset during or shortly following acute overload stress, or a history of gradual onset with chronic overload of the affected muscle; (2) characteristic patterns of pain that are referred from myofascial TPs; (3) weakness and restriction in the stretch range of motion of the affected muscle; (4) a taut, palpable band in the affected muscle; (5) exquisite, focal tenderness to digital pressure in the band of taut muscle fibers—the trigger point; (6) a local twitch response elicited through snapping palpation or needling of the tender spot or trigger point; (7) the reproduction of the patient's pain complaint by pressure on, or needling of, the tender spot or trigger point; and (8) the elimination of symptoms by therapy directed specifically to the affected muscles. Individuals typically will not meet all of the above criteria, however, the existence of one or more trigger points is a major symptom of all patients diagnosed with MPS.

A taut band (as mentioned in item (4) above) is a ropelike swelling found within the muscle, probably due to sustained shortening of muscle fibers. A twitch response (as mentioned in item (6) above) is a transient contraction of the muscle fibers of the taut band containing a TP. The twitch response can be elicited by snapping palpation of the trigger point, or more commonly by precise needling. A TP is typically identified by application of digital pressure or needling of the tender spot (typically by application of sustained pressure for 6-60 seconds), resulting in induction or reproduction of some or all of the patient's pain complaint.

Minor or secondary symptoms of MPS may include numbness, dizziness, headaches, concentration and memory problems, sleep disorders, fluid retention, balance problems, and stiffness.

As with the above syndromes, there is no single diagnostic test that confirms the diagnosis of MPS.

Multiple Chemical Sensitivity (MCS) Syndrome. Multiple chemical sensitivity syndrome has been described under various names since the 1940s. A 1999 case definition of MCS describes it as a chronic condition with reproducible symptoms involving multiple organ systems whose symptoms are produced by low levels of exposure to multiple, chemically unrelated substances and improve or resolve upon removal of the chemical agents (Bartha, L., et al., Arch Environ Health 1999: 54: 147-9). Self-reported high levels of reactivity to chemical exposure have been found in patients with CFS in comparison to healthy control subjects. (Friedberg F., et al, Proceedings of the American Association of Chronic Fatigue Syndrome Research Conference; 1994 October; Fort Lauderdale (Fla.); 1994). In a sample of 33 Gulf War veterans with CFS, 42% had concurrent MCS and 6% had FMS (Pollet, C., et al, J. Med 1998; 29:101-13). In another sample of 100 patients with MCS, it was found that 88% met criteria for CFS and 49% met the criteria for FMS (Donnay, A., Ziem, G. Proceedings of the American Association of Chronic Fatigue Syndrome Research Conference; 1998 October; Cambridge (Mass.), 1998).

A diagnosis of MCS typically involves screening as described above for the other related syndromes. For examples, a clinician will typically gather a patient history, conduct a detailed physical examination as well as conduct laboratory tests to rule out exclusionary medical conditions. Additionally, a medical questionnaire is typically administered in which subjects are asked if they had symptoms of feeling ill from a low level of exposure to two or more listed chemical agents that affected two “or more organ systems (Bartha, L, 1999, ibid). Subjects are diagnosed with MCS if they report positively to the above inquiry. Thus, in accordance with the present invention, a major symptom of MCS is an ill feeling or symptoms that occur reproducibly in two or more organs in response to low levels of exposure to at least two unrelated chemicals that improves or resolves when the chemicals are removed. Symptoms typically occur in one of three categories: central nervous system symptoms, respiratory and mucosal irritation, or gastrointestinal problems and include fatigue, difficulty concentrating, depression, memory loss, weakness, dizziness, headache, heat intolerance, and arthralgia. Exposures include aerosol air freshener, aerosol deodorant, after shave, asphalt pavement, cigar smoke, cigarette smoke, cologne, diesel exhaust, diesel fuel, dry cleaning fluid, floor cleaner, furniture polish, garage fumes, gasoline exhaust, hair spray, insect repellant, insecticide spray, laundry detergent, marking pens, nail polish, nail polish remover, oil-based paint, paint thinner, perfumes in cosmetics, public restroom deodorizers, shampoo, tar fumes from roof or road, tile cleaners, varnish, shellac and lacquer (Lax M B, Henneberger P K. Arch Environ Health 1995; 50:425-31).

Gulf War Syndrome (GWS). Yet another condition treatable in accordance with the present invention is GWS. GWS is the name given to a variable combination of unexplainable psychological and physical complaints experienced by veterans of the Persian Gulf War. Symptoms may include aching muscles, spasms, fatigue, irritability, thick saliva, weight loss, diarrhea, skin rashes, memory loss, dizziness, peripheral numbness, sleep disturbance, along with chronic fevers, labored breathing, and headaches. There is currently no well-accepted explanation for the symptoms experienced by Gulf War veterans, although several theories exist including exposure to chemical warfare agents, psychological factors, and exposure to other chemicals such as depleted uranium. In accordance with the invention, a subject that is a veteran of the Gulf War and that has experienced one or more of the above symptoms, each of which is considered herein as a major symptom, is considered to have GWS.

A subject with GWS may be one who is experiencing GWS 1, which involves symptoms such as sleep and memory disturbance. A subject with GWS may also be one who is experiencing GWS 2, whose symptoms include confusion and dizziness. Yet another subject with GWS may be one experiencing GWS 3, whose symptoms include muscle and joint pain. The three distinct syndromes were identified by a medical team in the U.S. using a statistical technique called factor analysis, which reveals unusual clusters of symptoms (Kang H K, et al., Am J. Epidemiol. 2003; 157:141-8).

Formulations

While limited success has been associated with prior therapies to date for FMS and related rheumatic conditions as described above, the inventors have discovered that administration of a formulation comprising trimetazidine is surprisingly effective in treating symptoms associated with FMS. As can be seen in the supporting examples, patients suffering from symptoms such as widespread pain, trigger point pain and tenderness, depression, dizziness, impaired concentration, symptoms of irritable bowel syndrome, fatigue, headache, impaired memory, and sleep disturbance, among others, reported significant, if not remarkable and sustained improvement in their symptoms after a course of therapy with trimetazidine.

The compositions, combinations, and methods of the invention will now be described in detail in the sections that follow.

Trimetazidine

Trimetazidine (1-(2,3,4-trimethyloxybenzyl)piperazine) possesses the structure shown below. 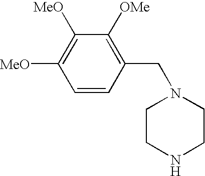
Trimetazidine was first marketed in 1978. Trimetazidine is a metabolic antianginal and anti-ischaemic agent that is used in the treatment of patients with coronary heart disease and stable angina, although its mechanism of action is not fully understood (Morin, D. et al., Br. J Pharmacol. 1998, April; 123(7): 1385-94; Makolkin, V. I.; Osadchiy, K. K., Clin. Drug Invest. 2004; 24 (12); 731-738).

Trimetazidine is commercially available in generic form from various commercial suppliers. In certain instances, trimetazidine may be referred to as generic Vasterel®. Additional tradenames for trimetazidine include Trivedon 20 (Cipla Ltd, India), Feelnor (Incepta Pharmaceuticals Ltd, Bangledesh), and Flavedon (Serdia Pharmaceuticals, India). Trimetazidine is commercially available as a film coated tablet (20 mg), and as a modified release formula (35 mg), commonly referred to as “trimetazidine MR”, or as “Vasterel® MR” (Servier, France). The modified release formula contains a hydrophilic hypromellose matrix that forms a gel upon contact with gastrointestinal fluids (Meurin, P., Henane, T. Heart and Metabolism, 2003; 21:29-31). The active agent, trimetazidine, may, in one or more embodiments of the invention, be used in either of the above forms, among others.

As stated previously, a reference to trimetazidine, as with all of the drugs or active molecules described herein, or derivatives of trimetazidine, where applicable, is meant to encompass any and all enantiomers, mixtures of enantiomers including racemic mixtures, prodrugs, pharmaceutically acceptable salt forms, hydrates (e.g., monohydrates, dihydrates, etc.), different physical forms (e.g., crystalline solids, amorphous solids), metabolites, and the like. Commercially available trimetazidine is typically provided as its hydrochloride salt, either trimetazidine hydrochloride or trimetazidine dihydrochloride, although any pharmaceutically acceptable salt form of trimetazidine may be used in the method of the invention.

Alternatively, trimetazidine for pharmaceutical use may be synthesized, e.g., using conventional chemical synthetic techniques, by a toll manufacturer in accordance with good manufacturing practices (GMP) standard operating procedures for pharmaceuticals. Such toll manufacturers suitable for preparing bulk trimetazidine for pharmaceutical applications in accordance with GMP practices include Davos Chemical (New Jersey), Carbogen Laboratories AG (Switzerland) and Hovione SA (Lisbon), and the like.

Also useful in the method of the invention are substituted derivatives of trimetazidine, such as those trimetazidine compounds described in U.S. Pat. No. 5,283,246, the contents of which is incorporated herein by reference in its entirety.

Additional Active Agents

In addition to trimetazidine, a therapeutic composition of the invention may optionally include a therapeutically effective amount of one or more additional active agents, herbs, vitamins, minerals, or other supplements useful in treating the subject rheumatic condition. If desired, in addition to trimetazidine, a composition of the invention may, but does not necessarily, also include one or more of the following: antidepressants such as amitriptyline, duloxetine, fluoxetine, paroxetine, sertraline, venlafaxine, trazodone, and bupropion; muscle relaxants such as cyclobenzaprine, analgesics such as tramadol, naproxen, ibuprofen, and piroxicam; low dose tricyclic agents such as doxepin, desipramine, nortriptyline; anxioloytic agents such as alprazolam, clonazepam, and lorazepam; antihistamines such as astemizole, and loratadine; antihypotensive agents such as fludrocortisone; and beta blockers such as altenolol, among others.

Alternatively, a therapeutically effective amount of any one or more of the above active agents may be co-administered, as a separate dosage form, with trimetazidine for treatment of, e.g., FMS, CFS, MPS, or GWS.

Excipients/Additives

Optionally, the compositions of the invention may further comprise one or more pharmaceutically acceptable excipients to provide a pharmaceutical composition. Exemplary excipients include, without limitation, carbohydrates, starches (e.g., corn starch), inorganic salts, antimicrobial agents, antioxidants, binders/fillers, surfactants, lubricants (e.g., calcium or magnesium stearate), glidants such as talc, disintegrants, diluents, buffers, acids, bases, film coats, combinations thereof, and the like.

A composition of the invention may include one or more carbohydrates such as a sugar, a derivatized sugar such as an alditol, aldonic acid, an esterified sugar, and/or a sugar polymer. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol(glucitol)pyranosyl sorbitol, myoinositol, and the like.

Also suitable for use in the compositions of the invention are potato and corn-based starches such as sodium starch glycolate and directly compressible modified starch.

Further representative excipients include inorganic salt or buffers such as citric acid, sodium chloride, potassium chloride, sodium sulfate, potassium nitrate, sodium phosphate monobasic, sodium phosphate dibasic, and combinations thereof.

The composition may also include an antimicrobial agent, e.g., for preventing or deterring microbial growth. Non-limiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol; and combinations thereof.

A composition of the invention may also contain one or more antioxidants. Antioxidants are used to prevent oxidation, thereby preventing the deterioration of the drug(s) or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

Additional excipients include surfactants such as polysorbates, e.g., “Tween 20” and “Tween 80,” and pluronics such as F68 and F88 (both of which are available from BASF, Mount Olive, N.J.), sorbitan esters, lipids (e.g., phospholipids such as lecithin and other phosphatidylcholines, and phosphatidylethanolamines), fatty acids and fatty esters, steroids such as cholesterol, and chelating agents, such as EDTA, zinc and other such suitable cations.

Further, a composition of the invention may optionally include one or more acids or bases. Non-limiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of any individual excipient in the composition will vary depending on the role of the excipient, the dosage requirements of the active agent components, and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 5% to about 98% by weight, more preferably from about 15 to about 95% by weight of the excipient, with concentrations less than 30% by weight most preferred.

These foregoing pharmaceutical excipients along with other excipients are described in “Remington: The Science & Practice of Pharmacy”, 19^{th} ed., Williams & Williams, (1995), the “Physician's, Desk Reference”, 52^{nd} ed., Medical Economics, Montvale, N.J. (1998), and Kibbe, A. H., Handbook of Pharmaceutical Excipients, 3^{rd} Edition, American Pharmaceutical Association, Washington, D.C., 2000.

Delivery Forms

The compositions encompass all types of formulations and in particular those that are suited for oral administration, e.g., tablets, lozenges, capsules, syrups, oral suspensions, emulsions, granules, and pellets. Alternative formulations include aerosols, transdermal patches, gels, creams, ointments, suppositories, powders or lyophilates that can be reconstituted, as well as liquids, such as for use in an oral or parenteral product. Examples of suitable diluents for reconstituting solid compositions, e.g., prior to injection, include bacteriostatic water for injection, dextrose 5% in water, phosphate-buffered saline, Ringer's solution, saline, sterile water, deionized water, and combinations thereof. With respect to liquid pharmaceutical compositions, solutions and suspensions are envisioned.

In turning now to oral delivery formulations, tablets can be made by compression or molding, optionally with one or more accessory ingredients or additives. Compressed tablets are prepared, for example, by compressing in a suitable tabletting machine, the active ingredients in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) and/or surface-active or dispersing agent.

Molded tablets are made, for example, by molding in a suitable tabletting machine, a mixture of powdered compounds moistened with an inert liquid diluent. The tablets may optionally be coated or scored, and may be formulated so as to provide slow or controlled release of the active ingredients, using, for example, hydroxypropylmethyl cellulose in varying proportions to provide the desired release profile. Tablets may optionally be provided with a coating, such as a thin film, sugar coating, or an enteric coating to provide release in parts of the gut other than the stomach. Processes, equipment, and toll manufacturers for tablet and capsule making are well-known in the art.

Capsule formulations may utilize either hard or soft capsules, including gelatin capsules or vegetarian capsules such as those made out of hydroxymethylpropylcellulose (HMPC). One preferred type of capsule is a gelatin capsule. Capsules may be filled using a capsule filling machine such as those available from commercial suppliers such as Miranda International or employing capsule manufacturing techniques well-known in the industry, as described in detail in Pharmaceutical Capules, 2^{nd} Ed., F. Podczeck and B. Jones, 2004. Alternatively, capsule formulations may be prepared using a toll manufacturing center such as the Chao Center for Industrial Pharmacy & Contract Manufacturing, located at Purdue Research Park.

Formulations for topical administration in the mouth include lozenges comprising the active ingredients, generally in a flavored base such as sucrose and acacia or tragacanth and pastilles comprising the active ingredients in an inert base such as gelatin and glycerin or sucrose and acacia.

A pharmaceutical composition for topical administration may also be formulated as an ointment, cream, suspension, lotion, powder, solution, paste, gel, spray, aerosol or oil. Alternatively, the formulation may be in the form of a patch (e.g., a transdermal patch) or addressing such as a bandage or adhesive plaster impregnated with active ingredients and optionally one or more excipients or diluents. Topical formulations may additionally include a compound that enhances absorption or penetration of the ingredients through the skin or other affected areas, such as dimethylsulfoxidem bisabolol, oleic acid, isopropyl myristate, and D-limonene, to name a few.

For emulsions, the oily phase is constituted from known ingredients in a known manner. While this phase may comprise merely an emulsifier (otherwise known as an emulgent), it desirably comprises a mixture of at least one emulsifier with a fat and/or an oil. Preferably, a hydrophilic emulsifier is included together with a lipophilic emulsifier that acts as a stabilizer. Together, the emulsifier(s) with or without stabilizer(s) make up the so-called emulsifying wax, and the wax together with the oil and/or fat make up the so-called emulsifying ointment base which forms the oily dispersed phase of cream formulations. Illustrative emulgents and emulsion stabilizers include Tween 60, Span 80, cetostearyl alcohol, myristyl alcohol, glyceryl monostearate and sodium lauryl sulfate.

Formulations for rectal administration are typically in the form of a suppository with a suitable base comprising, for example, cocoa butter or a salicylate.

Formulations suitable for vaginal administration generally take the form of a suppository, tampon, cream, gel, paste, foam or spray.

Formulations suitable for nasal administration, wherein the carrier is a solid, include a coarse powder having a particle size, for example, in the range of about 20 to about 500 microns. Such a formulation is typically administered by rapid inhalation through the nasal passage, e.g., from a container of the powder held in proximity to the nose. Alternatively, a formulation for nasal delivery may be in the form of a liquid, e.g., a nasal spray or nasal drops.

Aerosolizable formulations for inhalation may be in dry powder form (e.g., suitable for administration by a dry powder inhaler), or, alternatively, may be in liquid form, e.g., for use in a nebulizer. Nebulizers for delivering an aerosolized solution include the AERx™ (Aradigm), the Ultravent® (Mallinkrodt), and the Acorn II® (Marquest Medical Products). A composition of the invention may also be delivered using a pressurized, metered dose inhaler (MDI), e.g., the Ventolin® metered dose inhaler, containing a solution or suspension of a combination of drugs as described herein in a pharmaceutically inert liquid propellant, e.g., a chlorofluorocarbon or fluorocarbon.

Formulations suitable for parenteral administration include aqueous and non-aqueous isotonic sterile solutions suitable for injection, as well as aqueous and non-aqueous sterile suspensions.

Parenteral formulations of the invention are optionally contained in unit-dose or multi-dose sealed containers, for example, ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the types previously described.

A formulation of the invention may also be a sustained release formulation, such that trimetazidine is released or absorbed slowly over time, when compared to a non-sustained release (immediate release) formulation. Sustained release formulations may employ pro-drug forms of the active agent, delayed-release drug delivery systems such as liposomes or polymer matrices, hydrogels, or covalent attachment of a polymer such as polyethylene glycol to the active agent.

In addition to the ingredients particularly mentioned above, the formulations of the invention may optionally include other agents conventional in the pharmaceutical arts and particular type of formulation being employed, for example, for oral administration forms, the composition for oral administration may also include additional agents as sweeteners, thickeners or flavoring agents.

Kits

Also provided herein is a kit or package containing trimetazidine, in packaged form, accompanied by instructions for use. The trimetazidine may be packaged in any manner suitable for administration, so long as the packaging, when considered along with the instructions for administration, dearly indicates the manner in which trimetazidine is to be administered.

For example, the kit comprises trimetazidine in unit dosage form, along with instructions for use. For example, such instructions may indicate that administration of trimetazidine is useful in the treatment of conditions such as one or more of the following: FMS, CGS, MPS, MCS and GWS. Trimetazidine may be packaged in any manner suitable for administration, so long as the packaging, when considered along with the instructions for administration, clearly indicates the manner in which the drug component is to be administered. For example, when trimetazidine is in oral dosage form, e.g., is in the form of a coated tablet, then the kit may comprise a sealed container of coated tablets, blister strips containing the tablets, or the like. Other preferred dosage forms include capsules, syrups and suspensions. Preferably, for oral dosage forms, it is recommended that trimetazadine be administered after meals.

Various embodiments according to the above may be readily envisioned, and would of course depend upon the particular dosage form, recommended dosage, intended patient population, and the like. The packaging may be in any form commonly employed for the packaging of pharmaceuticals, such as medication punch cards or blisters, and may utilize any of a number of features such as different colors, wrapping, tamper-resistant packaging, blister paks or strips, dessicants, and the like.

Method of Administration

As set forth above, methods of delivery include but are not limited to, oral, intra-arterial, intramuscular, intravenous, intranasal, and inhalation routes. A preferred delivery route is oral. Suitable modes of delivery will be apparent based upon the particular combination of drugs employed and their known administration forms.

More particularly, trimetazadine for use in treating a rheumatic condition such as FMS, CFS, MPS, MCS, and GWS, may be administered by any suitable route, including without limitation, oral, rectal, nasal, topical (including transdermal, aerosol, buccal and sublingual), vaginal, penile, parenteral (including subcutaneous, intramuscular, intravenous and intradermal) and pulmonary. The preferred route will, of course, vary with the condition and age of the recipient, and the particular condition being treated.

Trimetazadine may be administered in unit dosage form multiple times daily, but most preferably is administered once, twice or three times daily. In terms of patient compliance and ease of administration, such an approach is preferred over more frequent dosing, since patients are often adverse to taking multiple pills or capsule, often multiple times daily, over the duration of treatment. In instances in which the trimetazadine is co-administered along with another active agent as separate dosage forms, each of the different active agents may be administered simultaneously, sequentially in any order, or separately.

Dosages and Measurable Result

Therapeutic amounts can be empirically determined and will vary with the particular condition being treated, the subject, the particular formulation components, dosage form, and the like. The actual dose to be administered will vary depending upon the age, weight, and general condition of the subject as well as the severity of the condition being treated, along with the judgment of the health care professional.

Therapeutically effective amounts can be determined by those skilled in the art, and will be adjusted to the requirements of each particular case. Generally, a therapeutically effective amount of trimetazidine will range from about 5 milligrams to about 210 milligrams daily. Preferably, a therapeutically effective amount of trimetazadine for the treatment of any one or more of FMS, CFS, MPS, MCS, and GWS, is in a range from about 20 milligrams to about 100 milligrams daily, or even more preferably from about 40 milligrams to about 80 milligrams daily. Unit dosage forms of trimetazadine are typically available as 20 milligram (immediate release) and 35 milligram (sustained release) formulations, although additional dosage forms are envisioned.

Representative dosages of trimetazadine are typically selected from the group consisting of: from about 5-100 mg/twice daily, from about 10 to about 50 mg/twice daily, from about 20 to about 40 mg twice daily, from about 10-50 mg three times daily, from about 20 to about 40 mg three times daily, about 35 mg/three times daily, and about 35 mg/twice daily, among others.

Practically speaking, a unit dose of any given trimetazadine composition of the invention can be administered in a variety of dosing schedules, depending on the judgement of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, every other day, three times weekly, twice weekly, once weekly, twice monthly, once monthly, and so forth.

The duration of treatment will depend of course on the particular condition, its severity, the age and condition of the patient, and the like, and will be readily determined by one of skill in the art. Illustrative courses of therapy include 1 week, 2 weeks, 3, weeks, 4 weeks, 5 weeks, 6 weeks, 7 weeks, 8 weeks, 9 weeks, 10 weeks, 11 weeks, 12 weeks, 3.5 months, 4 months, 4.5 months, 5 months, 6 months, 9 months, a year, or longer as needed. Treatment is typically continued until at least a 50% improvement is effected in one or more major symptoms associated with the particular condition being treated.

For example, in treating a subject suffering from FMS, treatment is generally continued until at least a 50% improvement is effected, e.g., in widespread pain of at least three anatomical sites of the subject's body, or in trigger point tenderness. Additionally, improvement may also be noted in one or more minor symptoms experienced by a subject suffering from FMS, e.g., fatigue, irritable bowel syndrome, sleep disorder, chronic headaches, jaw pain, cognitive or memory impairment, post-exertional malaise and muscle pain, morning stiffness, menstrual cramping, numbness and tingling sensations, dizziness, or chemical sensitivities. Preferably, treatment is continued until the subject experiences an improvement of at least about 60%, and even more preferably, at least about 70%, and even more preferably at least about 80%, and even more preferably, 90% or greater in at least one major symptom associated with the condition, e.g., FMS, and additionally, experiences a degree, e.g., 50% or greater, of improvement in one or more associated minor symptoms. Most preferably, treatment is generally continued until substantial resolution of all symptoms is effected or until the patient reports (or the physician notes) either no further improvement, or only minor or insignificant improvement in the subject's remaining symptoms with continued therapy as described herein.

The foregoing applies to each of the rheumatic conditions described herein as treatable by administration of trimetazidine.

For example, in the instance of a subject suffering from CFS, treatment is effected until the subject reports a lessening of at least about 50% in the degree of persistent fatigue experienced. Preferably, treatment is continued until the subject experiences an improvement of at least about 60%, and even more preferably, at least about 70%, and even more preferably at least about 80%, and even more preferably, 90% or greater in the degree of persistent fatigue experienced. Preferably, therapeutic treatment with trimetazadine is also effect to provide improvement in one or more minor CFS-associated symptoms experienced by the subject, e.g., impaired memory or concentration, tender cervical or axillary lymph nodes in the neck region, sore throat, muscle pain, multi joint pain, headaches, unrefreshing sleep, and post-exertional malaise. Again, such improvement will typically be of a degree of at least about 50% improvement or greater. Ideally, treatment with trimetazadine is effective to result in improvement of at least about 60%, or at least about 70%, or even more preferably at least about 80%, or most preferably 90% or greater of said one or more minor symptoms.

In the instance of a subject suffering from MPS, treatment is effected until the subject reports a lessening of the degree of pain in one or more trigger points of at least about 50%. Preferably, treatment is continued untile the subject experiences an improvement of at least about 60%, and even more preferably, at least about 70%, and even more preferably at least about 80%, and even more preferably, 90% or greater in the degree of muscle pain experienced in one or more trigger points. Preferably, therapeutic treatment with trimetazadine is also effect to provide improvement in one or more minor MPS-associated symptoms experienced by the subject, e.g., numbness, dizziness, headache, concentration and memory impairment, sleep disorders, fluid retention, balance problems (instability), and stiffness. Again, such improvement will typically be of a degree of at least about 50% improvement or greater. Ideally, treatment with trimetazadine is effective to result in improvement of at least about 60%, or at least about 70%, or even more preferably at least about 80%, or most preferably 90% or greater of said one or more minor symptoms associated with MPS.

Treatment of a subject suffering from GWS or from MCS with trimetazadine is over a duration of time effective to result in a similar degree of improvement of associated major and preferably minor symptoms associated therewith as described above.

It is to be understood that while the invention has been described in conjunction with preferred specific embodiments, the foregoing description as well as the examples that follow are intended to illustrate and not limit the scope of the invention. Other aspects, advantages and modifications within the scope of the invention will be apparent to those skilled in the art to which the invention pertains.

### Abbreviations

The following is a list of abbreviations that are used herein.
FMS fibromyalgia syndrome
CFS chronic fatigue syndrome
MCS multiple chemical sensitivities
MPS myofascial pain syndrome
GWS Gulf War syndrome
CBC complete blood count
ESR erythrocyte sedimentation rate
CRP C-reative protein
ANA antinuclear antibody
OTC over the counter
NSAID non-steroidal anti-inflammatory drug
MR modified release
TP trigger point
DSM-IV Diagnostic and Statistical Manual of Mental Disorders
SCID structured clinical interview for DSM-IV

All articles, books, patents and other publications referenced herein are hereby incorporated by reference in their entireties.

### EXAMPLES

### Example 1

### Treatment of Human Subject Diagnosed with Figromyalgia with Trimetazadine

A 40 year old male presented with long-standing complaints (15 years) of widespread pain—headaches, upper and lower back pain, neck pain, elbow and knee pain bilaterally, calf pain bilaterally and jaw pain. General non-specific symptoms were fatigue, symptoms of irritable bowel syndrome (constipation-type), insomnia, cognitive and memory impairment, morning stiffness, dizziness, irritability and depression.

Physical Examination: A physical examination revealed tender points located at:
(i) the sub-occipital muscle insertions bilaterally, at the occiput
(ii) the lateral epicondyle at the right elbow
(iii) the medial fat pads of the knees, just proximal to the joint lines, bilaterally
(iv) the trapezius muscles, at the midpoint of the upper border of the muscles, bilaterally
(v) the supraspinatus muscles, at the origins, above the spine of the scapula near the medial border, bilaterally.

Laboratory Tests: The following laboratory investigations were conducted: CBC, ESR, CRP, Rheumatoid Factor, Thyroid Function Tests, ANA. These tests yielded no positive results. X-Rays were also taken and resulted in no positive findings.

Patient History of Prior Treatment: A patient history was taken. Prior treatments targeting pain relief were as follows. OTC analgesics such as paracetamol (also known as acetaminophen) NSAIDs and low dose codeine) provided no pain relief whatsoever. Prescription analgesics such as high dose codeine, tramadol and meperidine provided, at best, mild to moderate and very temporary relief and were frequently accompanied by unpleasant side effects. Trigger point injections with lidocaine or lidocaine mixed with triamcinolone (a corticosteroid) provided the best localized pain relief. However, these injections often had to be repeated at 2 to 4 week intervals, and were usually extremely painful procedures.

Prior treatments targeting other symptoms included the following. The subject had taken tegaserod for irritable bowel syndrome. Additional prior therapies included sertraline for depression, and zolpidem for insomnia. The subject has also taken proclorperazine for dizziness.

All of the above therapies were either minimally effective or completely ineffective.

Diagnosis: The subject was diagnosed with fibromyalgia syndrome.

Relevant Additional Medical History: The subject also has cardiac tachyarrhythmia (likely supraventricular tachycardia) with paroxysmal episodes of chest pains and syncope—currently in the process of being investigated.

Treatment Regime: The subject was administered trimetazidine (modified release formulation), 35 mg orally, twice daily, originally for treatment of his cardiac condition.

Results: Within 72 hours of starting treatment, there was a surprising and completely unexpected and vast improvement in all symptoms and signs of fibromyalgia. The benefits were first noticed within 24 hours of commencing therapy and further improved gradually thereafter. Positive results were as follows. The subject reported a “90% reduction” in the widespread pain, with minimal residual pain reported at the left medial knee. The subject additionally reported very significantly increased energy levels, very significantly improved sleep patterns (described as “uninterrupted and very restful”), markedly improved regularity of bowel habits, markedly increased clarity of thoughts and improvement in short and long term memory, and very improved mood and markedly reduced level of irritability.

Follow-un. After six (6) months of continuous use of trimetazidine (modified release) 35 mg, orally, twice daily, the patient has reported that he continues to experience the same marked improvement in the quality of his life, with infrequent episodes of pain at his trigger points, which usually occurs in reaction to stressful episodes or concurrent bacterial or viral infections and usually reduces once again to negligible levels after these circumstances or conditions resolve.

### Example 2

### Treatment of Human Subject Diagnosed with Fibromyalgia with Trimetazadine

A 35 year old female presented with long-standing complaints (10 years) of widespread pain—headaches, upper and lower back pain, neck pain, hip pain bilaterally, knee pain bilaterally and jaw pain. General non-specific symptoms were fatigue, symptoms of irritable bowel syndrome (constipation-type), insomnia, cognitive and memory impairment, morning stiffness, and depression.

Physical Examination: A physical examination revealed tender points located at:
(vi) the sub-occipital muscle insertions bilaterally, at the occiput
(vii) the medial fat pads of the knees, just proximal to the joint lines, bilaterally
(viii) the trapezius muscles, at the midpoint of the upper border of the muscles, bilaterally
(ix)
(x) the supraspinatus muscles, at the origins, above the spine of the scapula near the medial border, bilaterally.

Laboratory Tests: The following laboratory investigations were conducted: CBC, ESR, CRP, Rheumatoid Factor, Thyroid Function Tests, ANA. These tests yielded no positive results. X-Rays were also taken and resulted in no positive findings.

Patient History of Prior Treatment. A patient history was taken. Prior treatments targeting pain relief were as follows. OTC analgesics such as paracetamol (also known as acetaminophen) NSAIDs and low dose codeine) provided no pain relief whatsoever. Prescription analgesics such as high dose codeine, tramadol and meperidine provided, at best, mild to moderate and very temporary relief and were frequently accompanied by unpleasant side effects. Trigger point injections with lidocaine or lidocaine mixed with triamcinolone (a corticosteroid) provided the best localized pain relief. However, these injections often had to be repeated at 2 to 4 week intervals, and were usually extremely painful procedures.

Prior treatments targeting other symptoms included the following. The subject had taken tegaserod for irritable bowel syndrome. Additional prior therapies included sertraline for depression, and zolpidem for insomnia.

All of the above therapies were either minimally effective or completely ineffective.

Diagnosis: The subject was diagnosed with fibromyalgia syndrome.

Treatment Regime: The subject was administered trimetazidine (modified release formulation), 35 mg orally, twice daily.

Results: Within 24 hours of starting treatment, there was vast improvement in all symptoms and signs of fibromyalgia. Positive results were as follows. The subject reported a “95% reduction” in the widespread pain, with minimal residual pain reported at the left hip. The subject additionally reported very significantly increased energy levels, very significantly improved sleep patterns (described as “uninterrupted and very restful”), markedly improved regularity of bowel habits, markedly increased clarity of thoughts and improvement in short and long term memory, and very improved mood.

Follow-up. After four (4) months of continuous use of trimetazidine (modified release) 35 mg, orally, twice daily, the patient has reported that she continues to experience the same marked improvement in the quality of her life. She has had no relapse of symptoms to date.

### Example 3

### Treatment of Human Subject Diagnosed with Fibromyalgia with Trimetazadine

A 32 year old female presented with a 5 year history of complaints of widespread pain—headaches, upper and lower back pain, neck pain, hip pain bilaterally and knee pain bilaterally. General non-specific symptoms were fatigue, symptoms of irritable bowel syndrome (constipation-type), insomnia, morning stiffness and emotional liability.

Physical Examination: A physical examination revealed tender points located at:
(xi) the sub-occipital muscle insertions bilaterally, at the Occiput
(xii) the medial fat pads of the knees, just proximal to the joint lines, bilaterally
(xiii) the trapezius muscles, at the midpoint of the upper border of the muscles, bilaterally
(xiv) the greater trochanter, posterior to the trochanteric prominence, bilaterally
(xv) the supraspinatus muscles, at the origins, above the spine of the scapula near the medial border, bilaterally.

Laboratory Tests: The following laboratory investigations were conducted: CBC, ESR, CRP, Rheumatoid Factor, Thyroid Function Tests, ANA. These tests yielded no positive results. X-Rays were also taken and resulted in no positive findings.

Patient History of Prior Treatment. A patient history was taken. Prior treatments targeting pain relief were as follows. OTC analgesics such as paracetamol (also known as acetaminophen) NSAIDs and low dose codeine) provided negligible pain relief. Prescription analgesics such as high dose codeine, tramadol and meperidine provided, at best, mild to moderate and very temporary relief and were frequently accompanied by unpleasant side effects. Trigger point injections were refused by the patient.

Prior treatments targeting other symptoms included the following. The subject had taken tegaserod for irritable bowel syndrome. Additional prior therapies included zolpidem, midazolam, zopiclone, and chlorpheniramine for insomnia.

All of the above therapies were either minimally effective or completely ineffective.

Diagnosis: The subject was diagnosed with fibromyalgia syndrome.

Treatment Regime: The subject was administered trimetazidine (modified release formulation), 35 mg orally, twice daily.

Results: Within 24 hours of starting treatment, there was vast improvement in all symptoms and signs of fibromyalgia. Positive results were as follows. The subject reported a “95% reduction” in the widespread pain, with a residual mil headache. The subject additionally reported very significantly increased energy levels, very significantly improved sleep patterns (described as “uninterrupted and very restful”), markedly improved regularity of bowel habits, and very improved mood.

Follow-up. After four (4) months of continuous use of trimetazidine (modified release) 35 mg, orally, twice daily, the patient has reported that she continues to experience the same marked improvement in the quality of her life. She has experienced no relapse of symptoms to date.

### Example 4

### Treatment of Eight (8) Human Subjects Diagnosed with Fibromyalgia with Trimetazadine

The following is a synopsis of the clinical response of each of eight (8) patients diagnosed with FMS, each of whom received treatment with trimetazidine MR, 35 mg orally, twice daily, for a period of at least four (4) weeks

TABLE 1
Summary of Treatment of 8 FMS Patients With Trimetazidine

| | | | | | | RESPONSE TO | |
|---|---|---|---|---|---|---|---|
| | | AGE OF | | | RESPONSE TO | TRIMETAZIDINE | |
| | | ONSET OF | SUMMARY OF | | TRIMETAZIDINE | (Medium Term | |
| | | FMS | FMS SIGNS | PREVIOUS | (Short Term i.e. | i.e. over four | EXAMINATION |
| AGE | SEX | SYMPTOMS | & SYMPTOMS | TREATMENTS | within 72 hours) | (4) weeks) | FINDINGS |
| 40 yrs | Female | 16 yrs old | Widespread Pain | Muscle Relaxant | 90% reduction in | After four (4) | Sustained |
| | | | Multiple Severe | Antidepressants | widespread pain | months of daily | resolution of all |
| | | | Trigger Point Pain & | OTC Analgesics | and trigger point | use, patient | trigger point |
| | | | Tenderness - | Opiate Analgesics | pain. | reported a 95% | tenderness. |
| | | | especially at lateral | Trigger Point | Significantly | sustained | |
| | | | epicondyle & bilateral | Injections | improved mood | improvement of | |
| | | | suboccipital | Gastric Acid | and concentration. | symptoms. | |
| | | | Depression | Suppressants | Infrequent | Residual symptoms - | |
| | | | Dizziness | Vestibular | dizziness/dizzy | include mild acid | |
| | | | Impaired | Sedatives | spells | reflux and | |
| | | | Concentration | | 90% reduction in | occasional | |
| | | | Irritable Bowel | | IBS and Acid | infrequent | |
| | | | Syndrome | | Reflux Symptoms | headaches | |
| | | | Acid Reflux | | | | |

| | | | | | | RESPONSE TO | |
|---|---|---|---|---|---|---|---|
| | | AGE OF | | | RESPONSE TO | TRIMETAZIDINE | |
| | | ONSET OF | SUMMARY OF | | TRIMETAZIDINE | (Medium/Long Term | |
| | | FMS | FMS SIGNS | PREVIOUS | (Short Term i.e. | i.e. over four | EXAMINATION |
| AGE | SEX | SYMPTOMS | & SYMPTOMS | TREATMENTS | within 72 hours) | (4) weeks) | FINDINGS |
| 32 yrs | Female | 24 yrs old | Widespread Pain | Muscle Relaxant | 90% reduction in | After six (6) weeks | Sustained |
| | | | Trigger Point Pain & | Antidepressants | widespread pain | of daily use, patient | resolution of all |
| | | | Tenderness in mid & | OTC Analgesics | and trigger point | reported a dramatic | trigger point |
| | | | lower back & medial | Opiate Analgesics | pain. | (95%) sustained | tenderness. |
| | | | aspect of ® knee | Trigger Point | Significantly | improvement of | |
| | | | Severe headaches | Injections | improved mood | symptoms. | |
| | | | Fatigue | Vestibular | and concentration. | Residual symptoms | |
| | | | Depression | Sedatives | Infrequent | include occasional | |
| | | | Sleep Disturbance | Yoga | dizziness/dizzy | insomnia. | |
| | | | Dizziness | | spells | | |
| | | | Impaired | | Consistent restful | | |
| | | | Concentration | | sleep | | |
| | | | Disorientation | | | | |
| 36yrs | Female | 32 yrs old | Widespread Pain | Antidepressants | 90% reduction in | After seven (7) | Not Applicable |
| | | | No identifiable | OTC Analgesics | widespread pain | weeks of daily use, | |
| | | | Trigger Point Pain or | Opiate Analgesics | Significantly | patient reported a | |
| | | | Tenderness | Vestibular | improved mood | dramatic 95% | |
| | | | Joint Pain | Sedatives | and concentration. | sustained | |
| | | | Severe headaches | Physiotherapy | Infrequent | improvement of | |
| | | | Fatigue | Acupuncture | dizziness/dizzy | symptoms with no | |
| | | | Depression | Anti-Spasmodics | spells | specific | |
| | | | Sleep Disturbance | | Consistent restful | complaints. | |
| | | | Dizziness | | sleep | | |
| | | | Impaired Memory | | 70% reduction in | | |
| | | | Word Finding | | severity of Pre- | | |
| | | | Difficulty | | Menstrual Cramps | | |
| | | | Severe Pre- | | | | |
| | | | Menstrual Cramps | | | | |
| 37 yrs | Male | 19 yrs old | Widespread Pain | Antidepressants | 90% reduction in | After four (4) | Not Applicable |
| | | | Multiple Trigger | OTC Analgesics | widespread pain | weeks of daily use, | |
| | | | Points especially at | Muscle Relaxants | Significantly | patient reported a | |
| | | | the right wrist and | | improved mood | dramatic 90% | |
| | | | both knees | | and concentration. | sustained | |
| | | | Joint Pain | | Infrequent | improvement of | |
| | | | Fatigue | | dizziness/dizzy | symptoms with no | |
| | | | Depression | | spells | specific | |
| | | | Sleep Disturbance | | Consistent restful | complaints. | |
| | | | Dizziness | | sleep | | |
| | | | Impaired Memory | | Energetic feeling | | |
| | | | Word Finding | | | | |
| | | | Difficulty | | | | |
| 48 yrs | Female | 37 yrs old | Widespread Pain | Antidepressants | 90% reduction in | After nine (9) | Sustained |
| | | | Multiple Trigger | OTC Analgesics | widespread pain | weeks of daily use, | resolution of |
| | | | Points especially in | Muscle Relaxants | Significantly | patient reported a | Trigger point |
| | | | the upper and lower | Anti-Emetics | improved mood | 90% sustained | tenderness and |
| | | | back and hips | Opiate Analgesics | and concentration. | improvement of | markedly improved |
| | | | Spinal & Joint Pain | Sedatives and | Infrequent dizzy | symptoms with | affect |
| | | | Fatigue | Hypnotics | spells | residual infrequent | |
| | | | Depression/Nervousness | Analgesic | Consistent restful | episodes of joint | |
| | | | Sleep Disturbance | Adjuncts/ | sleep | pains only | |
| | | | Blurred Vision | Anticonvulsants | No Nervousness | | |
| | | | Nausea & Vomiting | | Absence of Nausea | | |
| | | | Impaired Memory | | and Vomiting | | |
| | | | Generalized stiffness | | Improved Memory | | |
| | | | and tightness of the | | 90% reduction of | | |
| | | | muscles, worse in | | feeling of | | |
| | | | the a.m. | | generalized | | |
| | | | | | tightness and | | |
| | | | | | stiffness of muscles | | |
| | | | | | Energetic feeling | | |
| 37 yrs | Female | 31 yrs old | Widespread Pain | Antidepressants | 90% reduction in | After five (5) | Sustained |
| | | | Multiple Trigger | OTC Analgesics | widespread pain | weeks of daily use, | resolution of |
| | | | Points esp. in the | Muscle Relaxants | Significantly | patient reported a | Trigger point |
| | | | upper chest, the | Anti-Emetics | improved mood | dramatic 95% | tenderness and |
| | | | lower back, hips an d | Opiate Analgesics | and memory | sustained | markedly improved |
| | | | knees & gen. joint | Sedatives and | Absence of | improvement of all | Affect |
| | | | pains | Hypnotics | dizziness/dizzy | symptoms and | |
| | | | Restless Leg | Analgesic | spells | specifically an | |
| | | | Syndrome | Adjuncts/ | Consistent restful | increased sense of | |
| | | | Headaches & Nausea | Anticonvulsants | sleep | well-being | |
| | | | Burning sensation of | | Absence of Nausea | | |
| | | | the upper limbs and | | 90% reduction in | | |
| | | | the soles of the feet | | feeling of | | |
| | | | bilaterally and | | generalized | | |
| | | | increased sensitivity | | tightness and | | |
| | | | to cold | | stiffness of muscles | | |
| | | | Depression/Impaired | | Energetic feeling | | |
| | | | Memory & Fatigue | | Increased alertness | | |
| | | | Sleep Disturbance | | and clarity of | | |
| | | | Generalized stiffness | | thinking | | |
| | | | and tightness of the | | | | |
| | | | muscles, worse in | | | | |
| | | | the a.m. | | | | |
| 26 yrs | Female | 20 yrs old | Widespread Pain | Antidepressants | 90% reduction in | After four (4) | Sustained |
| | | | Multiple Trigger | OTC Analgesics | widespread pain | weeks of daily use, | resolution of |
| | | | Points esp. in the | Muscle Relaxants | Significantly | patient reported a | trigger point |
| | | | right elbow, behind | Opiate Analgesics | improved mood | dramatic 95% | tenderness and |
| | | | the neck and upper | Sedatives and | and memory | sustained | markedly improved |
| | | | back bilaterally and | Hypnotics | Consistent restful | improvement of all | affect and energy |
| | | | gen. joint pains. | Analgesic | sleep | symptoms and | levels with an |
| | | | Headaches and | Adjuncts/ | 90% reduction in | specifically an | obvious marked |
| | | | Retro-bulbar pain | Anticonvulsants | feeling of | increased sense of | improvement in |
| | | | Difficulty walking | Yoga | generalized | well-being and in | physical |
| | | | due to pain in the | Trigger Point | tightness and | particular a | appearance |
| | | | soles of the feet | injections | stiffness of muscles | sustained increase | |
| | | | Depression/Impaired | Physiotherapy | Energetic feeling | in energy levels | |
| | | | Memory & Fatigue | (muscle | Increased alertness | | |
| | | | Sleep Disturbance | stretching) | and clarity of | | |
| | | | Irritable Bowel | Acid | thinking | | |
| | | | Syndrome & Chest | Suppressants | Markedly reduced | | |
| | | | Pain (Acid Reflux) | Acupuncture | symptoms of | | |
| | | | Feeling of | Homeopathic | Irritable Bowel | | |
| | | | generalized stiffness | treatment | Syndrome | | |
| | | | and tightness of the | | Absence of Chest | | |
| | | | muscles, worse in- | | Pain | | |
| | | | the a.m. | | | | |
| 54 yrs | Female | 32 yrs old | Widespread Pain in | OTC Analgesics | 90% reduction in | After ten (10) | Markedly |
| | | | particular in the | Muscle Relaxants | widespread pain | weeks of daily use, | improved energy |
| | | | lower back, knees | Sedatives and | 90% reduction in | patient reported a | levels |
| | | | and flanks | Hypnotics | irritability, along | 90 to 95% | |
| | | | bilaterally; often | Anxiolytics | with 90% | improvement of all | |
| | | | worse with exertion | Physiotherapy | improvement in | symptoms. | |
| | | | and at nights & | Laxatives and | mood and memory | A flare up of FMS | |
| | | | gen. joint pain | Anti-Spasmodics | Consistent restful | symptoms occurred | |
| | | | Dizziness | Analgesic | sleep | once with the | |
| | | | Headaches/Retrobulbar | Adjuncts/ | Energetic feeling | occurrence of | |
| | | | pain | Anticonvulsants | Increased alertness | kidney stone pain | |
| | | | Impaired | | and clarity of | (Ureteric Colic) but | |
| | | | Concentration & | | thinking | resolved | |
| | | | Irritable Moods | | 90% reduction in | completely on | |
| | | | Photosensitivity | | symptoms of | passage the kidney | |
| | | | Sleep Disturbance/ | | Irritable Bowel | stone | |
| | | | Fatigue & | | Syndrome | | |
| | | | Nervousness | | | | |
| | | | Irritable Bowel | | | | |
| | | | Syndrome | | | | |
| | | | Chest Pain | | | | |
| | | | Numbness of the | | | | |
| | | | upper limbs | | | | |
| | | | bilaterally | | | | |

What is claimed is:

1. A method of treating a rheumatic condition selected from the group consisting of, fibromyalgia syndrome, chronic fatigue syndrome, myofascial pain syndrome, and Gulf war syndrome, said method comprising administering to a mammalian subject suffering from said condition a therapeutically effective amount of trimetazidine over a duration of time effective to result in a diminution of one or more major symptoms associated with said condition.

2. The method of claim 1, wherein said administering is over a duration of time effective to result in at least a 50% improvement in said one or more major symptoms.

3. The method of claim 1, wherein said administering is over a duration of time effective to result in at least a 90% improvement in said one or more symptoms.

4. A method of treating fibromyalgia, comprising administering to a mammalian subject suffering from fibromyalgia syndrome a therapeutically effective amount of trimetazidine over a duration of time effective to result in a diminution of one or more major symptoms associated with said fibromyalgia, wherein one of said symptoms is widespread pain of at least three anatomical sites of the subject's body.

5. The method of claim 4, wherein said administering is over a duration of time effective to result in at least a 50% reduction in said widespread pain.

6. The method of claim 5, wherein said administering is over a duration of time effective to result in at least a 90% reduction in said widespread pain.

7. The method of claim 6, wherein said therapeutically effective amount of trimetazidine ranges from approximately 5 milligrams to 210 milligrams daily.

8. The method of claim 7, wherein said therapeutically effective amount of trimetazidine ranges from approximately 20 milligrams to 100 milligrams daily.

9. The method of claim 8, wherein said therapeutically effective amount of trimetazidine ranges from approximately 40 milligrams to 80 milligrams daily.

10. The method claims 9, wherein said administering is by oral or parenteral administration.

11. The method of claim 10, wherein trimetazidine is administered orally.

12. The method of claim 7, wherein administering trimetazidine over a duration of 72 hours is effective to result in at least a 50% reduction in said widespread pain.

13. The method of claim 8, wherein administering trimetazidine over a duration of 72 hours is effective to result in at least a 50% reduction in said widespread pain.

14. The method of claim 9, wherein administering trimetazidine over a duration of 72 hours is effective to result in at least a 50% reduction in said widespread pain.

15. The method of claim 4, wherein said administering is further effective to result in a diminution of one of more symptoms selected from the group consisting of: tender point pain, depression, dizziness, impaired concentration, irritable bowel syndrome, headache, fatigue, and sleep disturbance.

16. The method of claim 15, wherein said administering is further effective to result in an improvement of at least 50% of one or more symptoms selected from the group consisting of: trigger point pain and tenderness, depression, dizziness, impaired concentration, irritable bowel syndrome, headache, fatigue, and sleep disturbance.

17. A kit comprising:
trimetazidine in packaged form, and
instructions for administering trimetazidine for treatment of a condition selected from the group consisting of fibromyalgia syndrome, chronic fatigue syndrome, myofascial-pain syndrone, and Gulf War syndrome.

18. The kit of claim 17, wherein said trimetazidine is in a form selected from the group consisting of a tablet, syrup, suspension, and capsule.

19. Use of trimetazidine for treatment of a condition selected from fibromyalgia syndrome, chronic fatigue syndrome, myofascial syndrome, and Gulf War syndrome.

20. Use of trimetazidine for the manufacture of a medicament for treating a condition selected from fibromyalgia syndrome, chronic fatigue syndrome, myofascial syndrome, and Gulf War syndrome.
